(19) United States
(12) Patent Application Publication
(10) Pub. No.: US 2018/0279947 A1
(43) Pub. Date: Oct. 4, 2018

(54) WEARABLE DEVICE WITH INTEGRATED SENSORS

(71) Applicant: Sunil Kumar Ummat, Bellevue, WA (US)

(72) Inventor: Sunil Kumar Ummat, Bellevue, WA (US)

(21) Appl. No.: 15/474,318

(22) Filed: Mar. 30, 2017

(51) Int. Cl. A61B 5/00; A61B 5/0476; A61B 5/0402; A61B 7/04; A61B 5/1455; A61B 5/0205

(52) U.S. Cl. CPC A61B 5/4818; A61B 5/4812; A61B 5/0476; A61B 5/0402; A61B 2562/0219; A61B 5/14551; A61B 5/0205; A61B 5/6804; A61B 5/002; A61B 7/04

(57) ABSTRACT

In one example, a system includes at least one wearable device, having at least one set of integrated sensors and a memory HUB for collecting data from any one or more of the sensors included in the at least one set of sensors. The sensors included in the at least one set of sensors monitor sleep cycles of a patient, record data, and transmit the recorded data to the memory HUB for collecting data.

### TECHNICAL FIELD

The embodiments described herein pertain generally to medical devices and application program products to use those medical devices in, e.g., the field of sleep study.

### BACKGROUND

Sleep apnea is a condition in which a patient stops breathing at night due to airway blockage. Sleep apnea is a type of sleep disorder characterized by pauses in breathing or instances of shallow or infrequent breathing during sleep. It can lead to many long term effects if not treated including increased risk for stroke, heart attacks, premature dementia and Alzheimer's disease. This condition now afflicts at least 50 as claimed million adults in the U.S., according to the National Healthy Sleep Awareness Project. Sleep study is an important study required for correct diagnosis of sleep conditions such as sleep apnea. Public health and safety are threatened by the increasing prevalence of obstructive sleep apnea.

### SUMMARY

In one example embodiment, a wearable device for performing a comprehensive sleep study, includes at least one set of integrated sensors including any of microphone tracheal sensor, electro cardiograph (ECG) sensor, belt sensor, pulse oxygen sensor, accelerometer, gyroscope, chest palpitation sensor and a combination thereof; and a memory HUB for collecting data from any one or more of the sensors included in the at least one set of integrated sensors. The one or more sensors included in the at least one set of sensors monitor sleep cycles of a patient, record data, and transmit the recorded data to the memory HUB.

In another example embodiment, a system for performing a comprehensive sleep study includes at least one wearable device, having at least one set of integrated sensors and a memory HUB for collecting data from any one or more of the sensors included in the at least one set of sensors. The sensors included in the at least one set of sensors monitor sleep cycles of a patient, record data, and transmit the recorded data to the memory HUB.

In an example embodiment, the system includes a first wearable device having a first set of integrated sensors including any of microphone tracheal sensor, electro cardiograph (ECG) sensor, belt sensor, pulse oxygen sensor, accelerometer, gyroscope, chest palpitation sensor and a combination thereof.

In another example embodiment, the system further includes a second wearable device having a second set of integrated sensors including any of one or more electroencephalogram (EEG) sensors, an oxygen sensor and a combination thereof.

In yet another example embodiment, a non-transitory computer-readable medium stores instructions that, when executed, cause one or more wearable processor-enabled devices for performing a comprehensive sleep study comprising at least one set of sensors to monitor sleep cycles of a patient wearing the one or more processor-enabled devices by recording data including, at least: airway sounds, including snoring and apnea, and electrical activity of the patient's heart over a period of time; and transmit the recorded data to a memory HUB that collects data from any one or more of the sensors included in the at least one set of sensors.

In another example embodiment, a method for performing a comprehensive sleep study is disclosed. The method for performing a comprehensive sleep study using one or more processor-enabled wearable devices includes monitoring sleep cycles of a patient wearing the one or more processor-enabled devices having at least one set of sensors, by recording data using the at least one set of sensors including: airway sounds, including snoring and apnea, and electrical activity of the patient's heart over a period of time; and receiving the recorded data by a memory HUB by Bluetooth transmission; wherein any one or more of the sensors included in the at least one set of sensors communicates with the memory HUB that collects data from the at least one set of sensors.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

In the detailed description that follows, embodiments are described as illustrations only since various changes and modifications will become apparent to those skilled in the art from the following detailed description. The use of the same reference numbers in different figures indicates similar or identical items.

FIG. 1 shows an example prior art in-hospital sleep study system.

FIG. 2 shows an example prior art at-home sleep study system.

FIG. 3 shows an example system configuration overview in which one or more embodiments of a system for conducting sleep study may be implemented, in accordance with various embodiments described herein;

FIG. 4 shows an example system configuration overview in which one or more embodiments of a system for conducting sleep study may be implemented, in accordance with various embodiments described herein;

FIG. 5 shows an example of a first wearable device in which one or more embodiments of a system for conducting sleep study may be implemented, in accordance with various embodiments described herein;

FIG. 6 shows an example of a second wearable device in which one or more embodiments of a system for conducting sleep study may be implemented, in accordance with various embodiments described herein;

FIG. 7 shows an example processing flow of operations for implementing at least portions of a system for conducting sleep study using at least one wearable processor-enabled device, in accordance with various embodiments described herein;

FIG. 8 shows an example processing flow of operations for implementing at least portions of a system for conducting sleep study using two wearable processor-enabled devices, in accordance with various embodiments described herein;

FIG. 9 shows an example computing device by which various embodiments of the process of conducting sleep study described herein may be implemented.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

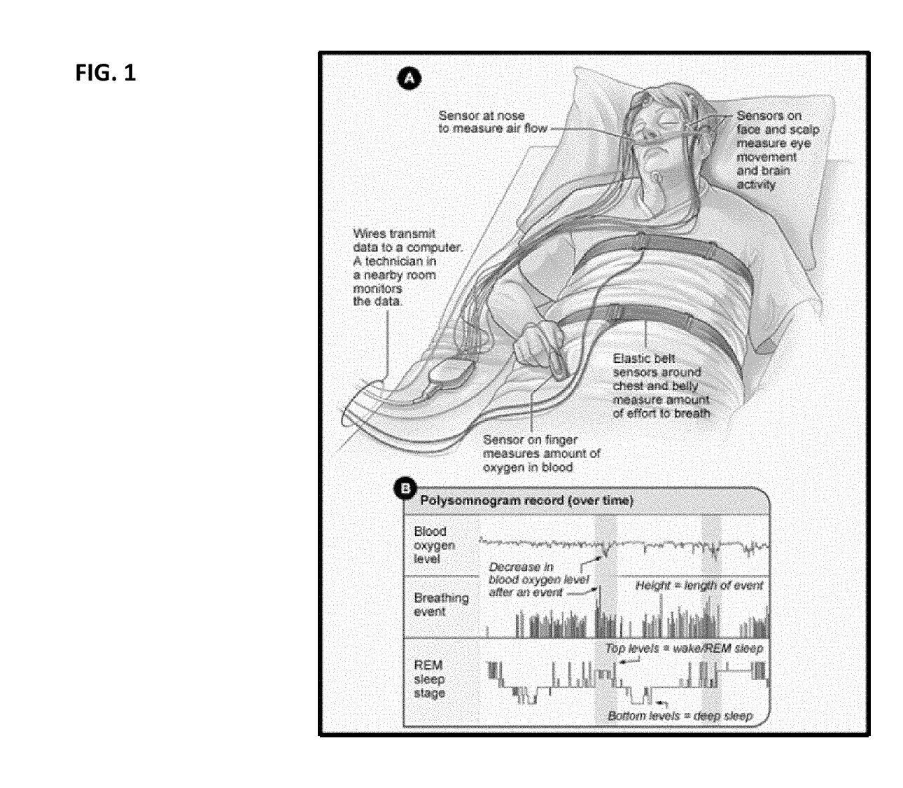

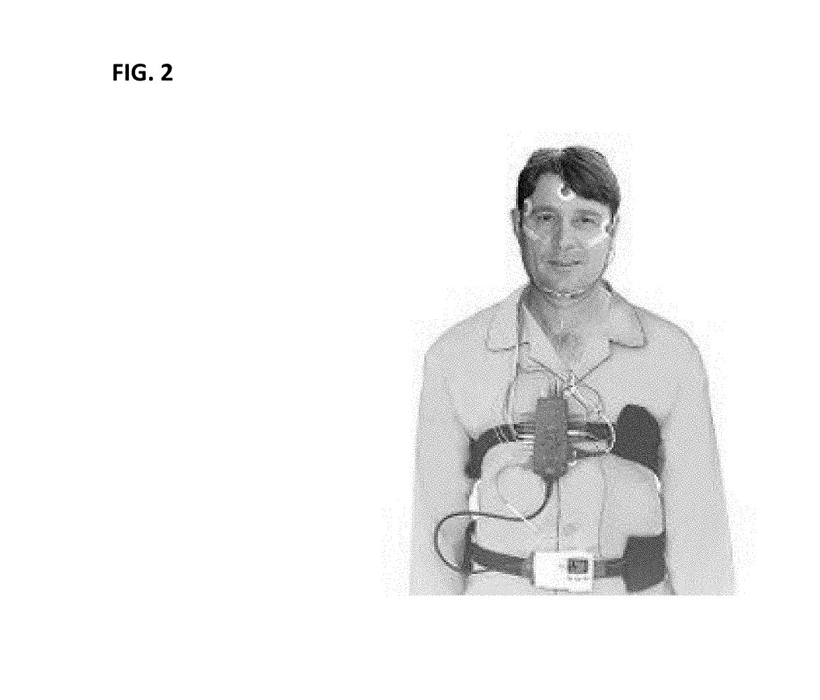

Many patients are hesitant to participate in a sleep study to evaluate their sleep habits and patterns because of reluctance or fear of an overnight hospital stay or extended stay in a sleep lab. FIG. 1 shows an example prior art in-hospital sleep study system used to perform a comprehensive sleep study. For various reasons including, e.g., patient comfort, availability of clinic resources, etc., home sleep studies are increasing in popularity. However, for home sleep study subjects or patients, the tangle of wires and cables that are part of the equipment as shown in FIG. 2 needed for home sleep study interfere with the patient's sleep causing inaccuracies in the collected data.

For example, inaccuracies can result from the patient being unable to get into a comfortable sleeping position due to the plurality of wires attached to probes or sensors on the patient's body and extending to a monitoring machine or computer. Thus, patients often end up sleeping on their backs for comfort during the test, which can actually aggravate sleep apnea.

Additionally, the currently available equipment for a home sleep study or screening does not enable a comprehensive sleep study that may clinically enable diagnosis of patients' sleep conditions. Patients may have to visit the hospital or sleep lab for proper diagnosis.

Described herein are embodiments of a system for performing a comprehensive sleep study that may include at least one wearable device, having at least one set of integrated sensors including any of microphone tracheal sensor, electro cardiograph (ECG) sensor, belt sensor, pulse oxygen sensor, accelerometer, gyroscope, chest palpitation sensor and a combination thereof; and a memory HUB that collects data from any one or more sensors included in the at least one set of sensors. The sensors included in the at least one set of sensors monitor sleep cycles of at least one patient, record data and transmit the recorded data to the memory HUB that collects data.

In an example embodiment, the system includes a first wearable device having a first set of integrated sensors including any of microphone tracheal sensor, electro cardiograph (ECG) sensor, belt sensor, pulse oxygen sensor, accelerometer, gyroscope, chest palpitation sensor and a combination thereof.

In one example embodiment, the system may also include a second wearable device having a second set of integrated sensors including any of one or more electroencephalogram (EEG) sensors, an oxygen sensor and a combination thereof, wherein the memory HUB collects data from any one or more sensors included in the first and second set of sensors.

Additional components of the system may further include a smartphone application for patients, a cloud database, and/or a web-based application for clinicians.

In one example embodiment, a wearable device for performing a comprehensive sleep study may include at least one set of integrated sensors including any of microphone tracheal sensor, electro cardiograph (ECG) sensor, belt sensor, pulse oxygen sensor, accelerometer, gyroscope, chest palpitation sensor and a combination thereof; and a memory HUB that collects data from any one or more sensors included in the at least one set of integrated sensors. One or more of the sensors monitors sleep cycles of a patient, records physiological data as the patient sleeps, and transmits the recorded data to the memory HUB that collects data from the various sensors.

In yet another example embodiment, the wearable device for performing a comprehensive sleep study includes a computer-readable medium integrated, attached, or otherwise associated therewith that stores instructions that, when executed, cause one or more associated sensors, processors, and/or process-enabled sensors to perform at least the following for conducting a sleep study: monitor sleep cycles of at least one patient wearing the device, by recording data including, at least: airway sounds, including snoring and apnea, and electrical activity of a user's heart over a period of time; and transmit the recorded data to a memory HUB that collects data from any one or more sensors included in the at least one set of sensors. One or more of the sensors communicates with the memory HUB by Bluetooth transmission or other known RFID technologies.

In another example embodiment, a method for performing a comprehensive sleep study is disclosed. The method for performing a comprehensive sleep study using one or more processor-enabled wearable devices includes monitoring sleep cycles of a patient wearing the one or more processor-enabled devices having at least one set of sensors, by recording data using the at least one set of sensors including, at least: airway sounds, including snoring and apnea, and electrical activity of the patient's heart over a period of time; and receiving the recorded data by a memory HUB by Bluetooth transmission; wherein any one or more of the sensors included in the at least one set of sensors communicates with the memory HUB that collects data from the at least one set of sensors.

Example embodiments described herein may enable comprehensive home sleep study producing results similar to the ones currently available in a hospital or sleep lab setting and may also be used to carry out comprehensive sleep study in a hospital or lab setting at the same time offering more patient comfort than the traditional comprehensive sleep study system.

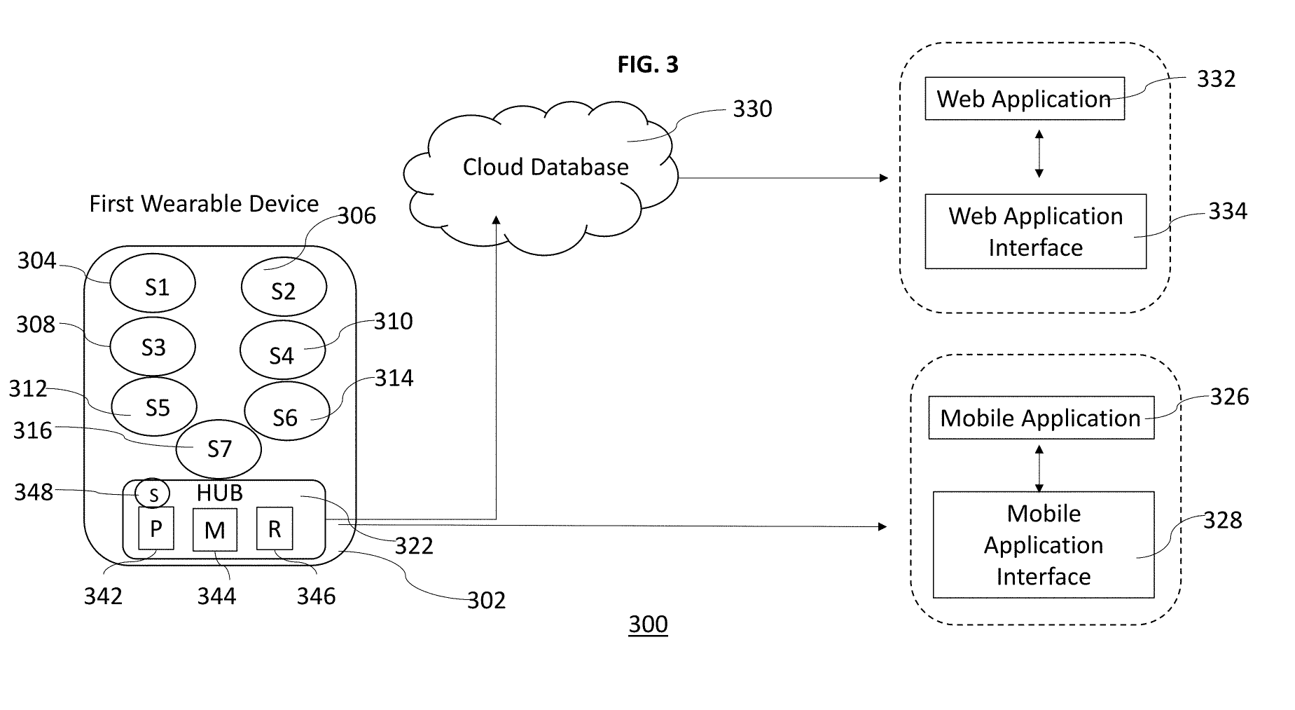

FIG. 3 shows an example system configuration in which one or more embodiments of a system for conducting a comprehensive sleep study may be implemented, in accordance with various embodiments described herein. By way of example, but not limitation, as depicted in FIG. 3 system 300 includes, at least, a first wearable device 302 having integrated therein a first set of sensors, and a memory HUB 322 that collects data from any one or more of the sensors included in the first set of sensors.

In an embodiment, the first wearable device 302 may be manufactured using a stretchable fabric for example, nylon, spandex, a combination of nylon and spandex, or Lycra. In an embodiment, the wearable device 302 may be a vest or a shirt made of stretchable fabric that may be pulled over reducing a need for a zipper or buttons. The first set of sensors used to conduct the sleep study may be integrated into the first wearable device 302, in an unobtrusive manner, unlike wires of conventional sleep study machinery, which can be entangled with the sleep study patient or, at least, cause noticeable physical discomfort. In an embodiment, the first set of sensors may be integrated in the first wearable device by adhering the sensors to the material of the wearable device, for example, by using an adhesive, adhesive strips or using fabric fasteners such as Velcro or stitching or using sensors that are woven into the fabric of the wearable device. In an example embodiment, the wires to the sensor may be woven through the fabric. In another example embodiment, the fabric itself may have stretch sensors within the fabric that collect and transmit data.

The first set of integrated sensors as depicted may include any of a microphone tracheal sensor or acoustic airway sensor 304, electro cardiograph (ECG) sensor 306, belt sensor 308, pulse oxygen sensor 310, accelerometer 312, gyroscope 314, chest palpitation sensor 316 and a combination thereof. These sensors are placed on the wearable device so as to enable sensing data from appropriate organs of a patient undergoing the study and, obviously, wearing the wearable device. For example, an ECG sensor 306 may be placed on the part of the first wearable device that would cover the patient's chest and, ostensibly, heart; a microphone tracheal sensor may be placed close to the thoracic cavity or the trachea of the patient.

Microphone tracheal sensor or acoustic airway sensor 304 monitors or measures airflow by listening and recording airway sounds such as in the trachea (or windpipe) of the patient wearing at least one or first wearable device 302. The recorded sounds, or sounds intended to be recorded, may include, but not be limited to, snoring and apnea.

ECG sensor 306 records electrical activity of the heart over a period of time and/or at predetermined time intervals. Examples of the recorded electrical activity include the subject's or patient's heart rate over a predetermined period of time or at predetermined intervals during the subject's or patient's sleep. ECG sensor 306 may be configured as a non-gel contact sensor, thus avoiding sticking sensors to a patient's body due to concerns of, e.g., skin allergies and discomfort associated with skin peeling.

Belt sensor 308 detects, measures, and records chest expansion as the subject or patient wearing the wearable device sleeps. These belt sensors may be stretch sensors that have an electrical distortion sensor. The stretch sensors are displaced by expansion of the chest. As the belt sensor is pulled apart from its baseline resting position, it creates an electrical effect that is transmitted to the sensor hub. The signals may then be analyzed for the extent of distortion and thereby the amount of chest expansion. These sensors record the amount of chest displacement in millimeters. This data may then be analyzed along with the data gathered by using pulse oximetry and other vital information to determine the relationship between the person's breathing and their level of oxygenation.

Pulse oxygen sensor 310 monitors and records oxygen saturation of the patient's blood and changes in blood volume in the skin. This sensor monitors and records changes in blood oxygenation by analyzing the difference in transmission of two wavelengths of light that are sent through a diode light source in contact with the patient's skin. The difference in light transmission that is recorded may be calibrated and compared to normal standards, resulting in a graphic or numerical value of blood oxygenation.

Accelerometer 312 and gyroscope 314 monitor and record sleeping position, changes in sleeping position, and stationary orientations of the patient while sleeping. An accelerometer is an electromechanical device that is used to measure acceleration and/or rotational movement. A gyroscope may measure rotational movement as well. These mechanical forces are then converted into electrical events that may be transmitted, recorded and analyzed by the software application. Positions described may include supine, left lateral, right lateral or prone.

Chest palpitation sensor 316 monitors and records the heart's electrical activity similar to ECG, by monitoring the heart's rhythm.

Memory HUB 322 may include any one or more of a rechargeable power source 342, a removable storage device 344 for data collection, for example, a fob, a USB drive or a SD card, and a radio-frequency identification (RFID) node 346 to facilitate communication with any of the sensors integrated within first wearable device 302. In at least one embodiment, memory HUB 322 may be adhered, stitched, or otherwise attached to the outer side of wearable device 302, may have a slim and non-intrusive form factor so as to minimize discomfort to the patient, and may include one or more sensors 348 that determine a body position of the patient while sleeping.

Body position sensor 348 may determine a bodily sleeping position of the patient using accelerometer 312. An accelerometer is an electromechanical device that is used to measure acceleration and/or rotational movement. These mechanical forces are then converted into electrical events that may be transmitted, recorded and analyzed by the software application. Positions described may include supine, left lateral, right lateral or prone. RFID node 346 facilitates Bluetooth connectivity, or some other known RFID communication technology as described above, with any of the sensors integrated within first wearable device 302.

In at least one other embodiment, memory HUB 322 may further comprise an interface 328 for interacting with at least one mobile application 326 that utilizes sleep analytics and trending features such as arousals due to breathing problems related to particular sleeping positions, arousals due to completion of sleep cycles, to provide a patient wearing at least one wearable device 302 with information about their sleep patterns. The arousals may be measured by analyzing multiple parameters including airflow measurement, EEG readings, gyroscope analysis, pulse oximetry etc. For example, airflow measurements may indicate cessation of airflow, and if observed along with drop in oxygen level may indicate apnea resulting in arousal from sleep. Similarly, sudden changes in EEG readings may indicate arousal if observed in combination with other factors such as increased heart rate or change in body position. Mobile application 326 and/or user interface 328 may be hosted on a smartphone, tablet, PC, or other processor-driven computing device that is local to the patient. In an embodiment, the sensors may connect wirelessly or via Bluetooth with a smartphone application 326 for data collection and transmission.

In at least one other embodiment, memory HUB 322 may itself host and/or operate a web based application 332 to present the data collected by memory HUB 322 in a standardized format and to enable multi-day sleep studies for diagnosis using full trending and analytics. The standardized format may enable the application to provide meaningful results from gathered data compared to other sleep tests currently available. In an embodiment, application 332 and/or user interface 334 may be hosted on a smartphone, tablet, PC, or other processor-driven computing device that may or may not be local to the patient.

In at least one other embodiment, memory HUB 322 may include a wireless communication node to upload the data collected by memory HUB 322 to a cloud database 330 for live monitoring. The live monitoring may be achieved by communicatively coupling memory HUB 322 to at least one sensor of the at least one wearable device 302. In an embodiment, the live monitoring may be achieved by communicatively coupling the cloud database 330 to one or more sensors of at least one wearable device 302 or to memory HUB 322.

HUB 322 may include diagnostic components to record and quantify various aspects of a sleep study, e.g. different stages of sleep, heart rate during different stages of sleep, respiratory disturbances during REM, NREM and/or MT events, and position of the patient and their correlation with different types of apnea such as obstructive apnea, central apnea, mixed apnea or hypopneas. The diagnostic components may record and quantify data gathered by sensors including pulse oximetry, body position, chest movement and other sensors described above. Examples of the various aspects of comprehensive home-studied sleep include any of time of onset of sleep, sleep efficiency, sleep latency, REM latency, wake after sleep onset (WASO), time and percentage in each sleep stage, any breathing irregularities, for example, apneas which are defined as temporary cessation of breathing especially during sleep, arousals, cardiac rhythm abnormalities, body position, oxygen saturation and a combination thereof.

Once the data regarding measurements of the aforementioned sleep aspects are acquired, for a period of time and/or at predetermined time intervals, any diversions from the expected values may be used by the analytics system to assess aspects of the patient's sleep. For example, time of onset of sleep may be measured by measuring electrical activity using one or more ECG sensors 306, as the electrical activity signals are different when a patient is awake and when the patient is asleep. Sleep efficiency may be measured using various mathematical formulae that include a ratio of total sleep time to time spent in bed along with many other factors affecting sleep as well as sleep discontinuity. For example, sleep efficiency %=(total sleep time/total time in bed)×100. as claimed Other sleep indices may be measured from the gathered data including but not limited to sleep latency, REM latency, wake after sleep onset (WASO), time and percentage in each sleep stage, etc. Breathing irregularities may be measured by using measurements from either chest palpitation sensor 316 or ECG sensor 306. Arousals may be detected using cardiac rhythm abnormalities which may be measured using ECG sensor 306. The accelerometer 312 and/or gyroscope 314 may measure body position as well as body movement of the patient during sleep. The oxygen saturation levels measured by using pulse oxygen sensor 310 integrated in the first wearable device 302 and may also indicate breathing problems during sleep.

For example, normal blood oxygen level is generally 96-97%. Any dips in oxygen saturation measured by pulse oxygen sensor 310 below this level may indicate breathing problems during the patient's sleep. As oxygen saturation in blood decreases, carbon dioxide levels in the blood increase, forcing the heart to pump more and more blood to oxygenate the blood. This activity also simultaneously causes the patient to breathe faster, which may be measured by microphone tracheal sensor or acoustic airway sensor 304, resulting in increased movement in the chest and abdomen and may be measured by chest palpitation sensors 316 and/or belt sensor 308. A continuous increase in the movement of the patient's chest and/or abdomen may result in the patient waking, also known as arousal, which may be detected by disruptive measurements taken by ECG sensor 306. This awakening along with the factors causing it may be recorded and quantified by the diagnostic components comprising data acquisition and analytics system to quantify the occurrence of such events during the sleep study, for example, number of awakenings related to dips in the oxygen saturation per hour, different stages of sleep, heart rate during different stages of sleep, respiratory disturbances during REM, NREM and/or MT events, and position of the patient and their correlation with different types of apnea such as obstructive apnea, central apnea, mixed apnea or hypopneas, etc.

In at least one embodiment, system 300 may include more than one wearable devices wherein each wearable device may include different or additional sensors than the ones described above.

Thus, FIG. 3 shows an exemplary embodiment of system 300 including, at least, one wearable device 302 having integrated therein at least one set of sensors and memory HUB 322 that collects, stores, and or transmits data from any one or more of the sensors included in the at least one set of sensors.

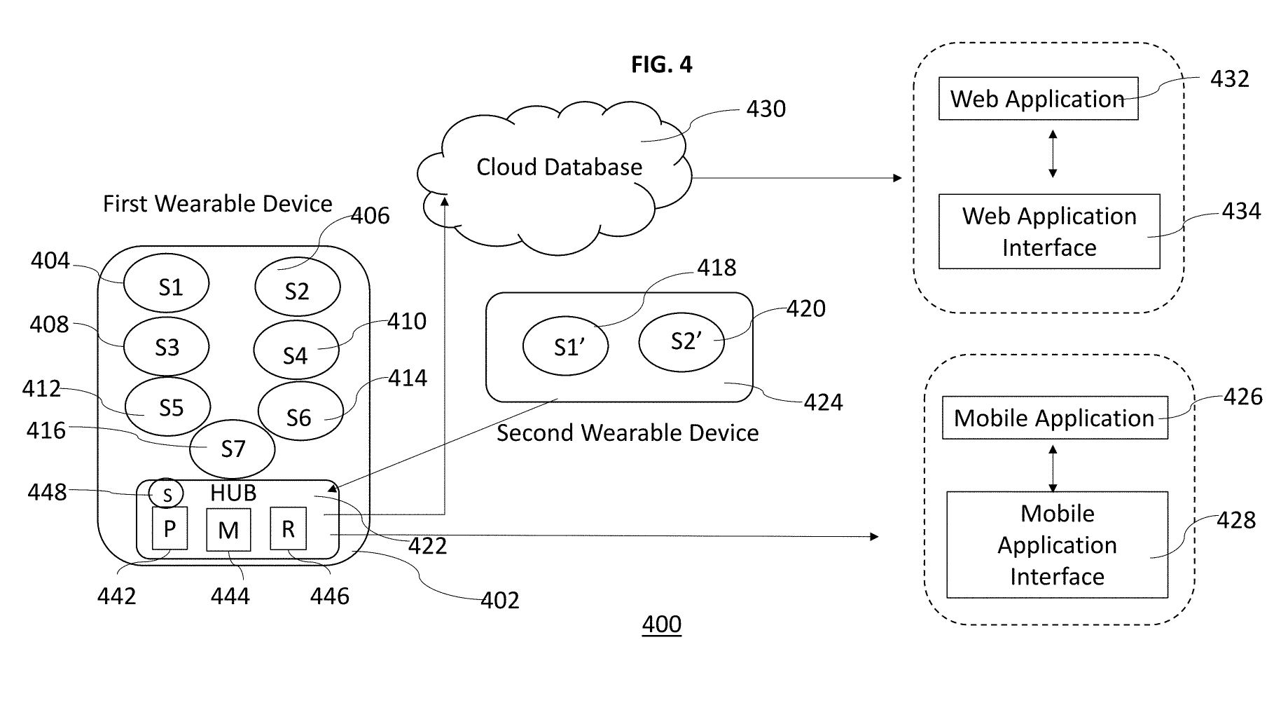

FIG. 4 shows an example system configuration in which one or more embodiments of a system for conducting a comprehensive sleep study may be implemented, in accordance with various embodiments described herein. By way of example, but not limitation, as depicted in FIG. 4 system 400 includes, at least, a first wearable device 402 having integrated therein a first set of sensors, a second wearable device 424 comprising a second set of integrated sensors and a memory HUB 422 that collects data from any one or more of the sensors included in the first and/or second set of sensors.

In an embodiment, the first wearable device 402 may be manufactured using a stretchable fabric for example, nylon, spandex, a combination of nylon and spandex, or Lycra. In an embodiment, the wearable device 402 may be a vest or a shirt made of stretchable fabric that may be pulled over reducing a need for a zipper or buttons. The first set of sensors used to conduct the sleep study may be integrated into the first wearable device 402, in an unobtrusive manner, unlike wires of conventional sleep study machinery, which can be entangled with the sleep study patient or, at least, cause noticeable physical discomfort. In an embodiment, the first set of sensors may be integrated in the first wearable device by adhering the sensors to the material of the wearable device, for example, by using an adhesive, adhesive strips or using fabric fasteners such as Velcro or stitching or using sensors that are woven into the fabric of the wearable device. In an example embodiment, the wires to the sensor may be woven through the fabric. In another example embodiment, the fabric itself may have stretch sensors within the fabric that collect and transmit data.

The first set of integrated sensors as depicted may include any of a microphone tracheal sensor or acoustic airway sensor 404, electro cardiograph (ECG) sensor 406, belt sensor 408, pulse oxygen sensor 410, accelerometer 412, gyroscope 414, chest palpitation sensor 416 and a combination thereof. These sensors are placed on the wearable device so as to enable sensing data from appropriate organs of a patient undergoing the study and, obviously, wearing the wearable device. For example, an ECG sensor 406 may be placed on the part of the first wearable device that would cover the patient's chest and, ostensibly, heart; a microphone tracheal sensor or acoustic airway sensor may be placed close to the thoracic cavity or the trachea of the patient.

Microphone tracheal sensor or acoustic airway sensor 404 measures airflow by listening and recording airway sounds such as in the trachea (or windpipe) of the patient wearing at least first wearable device 402. The recorded sounds, or sounds intended to be recorded, may include, but not be limited to, snoring and apnea.

ECG sensor 406 records electrical activity of the heart over a period of time and/or at predetermined time intervals. Examples of the recorded electrical activity include the subject's or patient's heart rate over a predetermined period of time or at predetermined intervals during the subject's or patient's sleep. ECG sensor 406 may be configured as a non-gel contact sensor, thus avoiding sticking sensors to a patient's body due to concerns of, e.g., skin allergies and discomfort associated with skin peeling.

Belt sensor 408 detects, measures, and records chest expansion as the subject or patient wearing the wearable device sleeps. These belt sensors may be stretch sensors that have an electrical distortion sensor. The stretch sensors are displaced by expansion of the chest. As the belt sensor is pulled apart from its baseline resting position, it creates an electrical effect that is transmitted to the sensor hub. The signals may then be analyzed for the extent of distortion and thereby the amount of chest expansion. These sensors record the amount of chest displacement in millimeters. This data may then be analyzed along with the data gathered by using pulse oximetry and other vital information to determine the relationship between the person's breathing and their level of oxygenation.

Pulse oxygen sensor 410 monitors and records oxygen saturation of the patient's blood and changes in blood volume in the skin. This sensor monitors and records changes in blood oxygenation by analyzing the difference in transmission of two wavelengths of light that are sent through a diode light source in contact with the patient's skin. The difference in light transmission that is recorded may be calibrated and compared to normal standards, resulting in a graphic or numerical value of blood oxygenation.

Accelerometer 412 and gyroscope 414 monitor and record sleeping position, changes in sleeping position, and stationary orientations of the patient while sleeping. An accelerometer is an electromechanical device that is used to measure acceleration and/or rotational movement. A gyroscope may measure rotational movement as well. These mechanical forces are then converted into electrical events that may be transmitted, recorded and analyzed by the software application. Positions described may include supine, left lateral, right lateral or prone.

Chest palpitation sensor 416 monitors and records the heart's electrical activity similar to ECG, by monitoring the heart's rhythm.

As depicted in FIG. 4, system 400 may further, or alternatively, include second wearable device 424 comprising a second set of integrated sensors. By way of example, but not limitation, the second wearable device 424 may be implemented as, for example, a headband or a cap comprising a second set of integrated sensors. In an embodiment, the second set of sensors may be integrated in the second wearable device by adhering the sensors to the material of the wearable device, for example, by using an adhesive, adhesive strips or using fabric fasteners such as Velcro, or stitching or using sensors that are woven into the fabric of the second wearable device 424.

The second set of integrated sensors included in second wearable device 424 as depicted may include any of one or more electroencephalogram (EEG) sensors 418, an oxygen sensor 420, or a combination thereof. The EEG sensors 418 monitor and record electrical activity in the patient's brain, and the oxygen sensor 420 monitors and records oxygen saturation in the patient's blood in a non-invasive manner. The EEG sensor measures voltage fluctuations resulting from ionic current within the neurons of the brain. These voltage fluctuations may be amplified, recorded and analyzed by the software application. To record the patient's brain activity during the sleep study, the one or more EEG sensors 418 may be placed on the headband or cap that may fit closer to the patient's scalp. Similarly, to monitor and record oxygen saturation in human blood non-invasively through skin, the oxygen sensor 420 may be placed close to the skin on the patient's forehead. Thus, the implementation of the system, including both the first and second wearable devices, may avoid attaching any devices or sensors to any part of the mouth or nose, avoiding impedances to the patient's sleep.

In an embodiment, at least one of the second set of sensors may communicate with one or both of memory HUB 422 attached to the first wearable device 402 or a smartphone application 426. RFID technologies facilitating such communication may be implemented by, e.g., adding a RFID tag to any one or more sensors included in the first and/or second set of sensors for automatic identification and data capture working in concert with the RFID node included in memory HUB 422 or a smartphone.

Memory HUB 422 may include any one or more of a rechargeable power source 442, a removable storage device 444 for data collection, for example, a fob, a USB drive or a SD card, and a radio-frequency identification (RFID) node 446 to facilitate communication with any of the sensors integrated within first wearable device 402 and second wearable device 424. In at least one embodiment, memory HUB 422 may be adhered, stitched, or otherwise attached to the outer side of wearable device 402, may have a slim and non-intrusive form factor so as to minimize discomfort to the patient, and may include one or more sensors 448 that determine a body position of the patient while sleeping.

Body position sensor 448 may determine a bodily sleeping position of the patient using accelerometer 412. An accelerometer is an electromechanical device that is used to measure acceleration and/or rotational movement. These mechanical forces are then converted into electrical events that may be transmitted, recorded and analyzed by the software application. Positions described may include supine, left lateral, right lateral or prone. RFID node 446 facilitates Bluetooth connectivity, or some other known RFID communication technology as described above, with any of the sensors integrated within first wearable device 402 and second wearable device 424.

In at least one other embodiment, memory HUB 422 may further comprise an interface 428 for interacting with at least one mobile application 426 that utilizes sleep analytics and trending features such as arousals due to breathing problems related to particular sleeping positions, arousals due to completion of sleep cycles, to provide a patient wearing either of wearable devices 402 and 424 with information about their sleep patterns. The arousals may be measured by analyzing multiple parameters including airflow movement, EEG readings, gyroscope analysis, pulse oximetry etc. For example, airflow measurements may indicate cessation of airflow, and if observed along with drop in oxygen level may indicate arousal from sleep. Similarly, sudden changes in EEG readings may indicate arousal if observed in combination with other factors such as increased heart rate or change in body position. These parameters may also help determination of different types of breathing problems that can arise during a sleep cycle, e.g. obstructive sleep apnea, upper airway resistance syndrome, primary snoring, etc. Mobile application 426 and/or user interface 428 may be hosted on a smartphone, tablet, PC, or other processor-driven computing device that is local to the patient. In an embodiment, the sensors may connect wirelessly or via Bluetooth with a smartphone application 426 for data collection and transmission.

In at least one other embodiment, memory HUB 422 may itself host and/or operate a web based application 432 to present the data collected by memory HUB 422 in a standardized format and to enable multi-day sleep studies for diagnosis using full trending and analytics. The standardized format may enable the application to provide meaningful results from gathered data compared to other sleep tests currently available. In an embodiment, application 432 and/or user interface 434 may be hosted on a smartphone, tablet, PC, or other processor-driven computing device that may or may not be local to the patient.

In at least one other embodiment, memory HUB 422 may include a wireless communication node to upload the data collected by memory HUB 422 to a cloud database 430 for live monitoring. The live monitoring may be achieved by communicatively coupling memory HUB 422 to at least one sensor of first wearable device 402. In an embodiment, the live monitoring may be achieved by communicatively coupling the cloud database 430 to one or more sensors of at least one of first wearable device 402 and second wearable device 424, or to memory HUB 422.

HUB 422 may include diagnostic components to record and quantify various aspects of a sleep study, e.g. different stages of sleep, heart rate during different stages of sleep, respiratory disturbances during REM, NREM and/or MT events, and position of the patient and their correlation with different types of apnea such as obstructive apnea, central apnea, mixed apnea or hypopneas. The diagnostic components may record and quantify data gathered by sensors including pulse oximetry, body position, chest movement, EEG and other sensors described above. Examples of the various aspects of home-studied sleep include any of time of onset of sleep, sleep efficiency, sleep latency, REM latency, wake after sleep onset, time and percentage in each sleep stage, any breathing irregularities, for example, apneas which are defined as temporary cessation of breathing especially during sleep, arousals, cardiac rhythm abnormalities, body position, oxygen saturation and a combination thereof.

Once the data regarding measurements of the aforementioned sleep aspects are acquired, for a period of time and/or at predetermined time intervals, any diversions from the expected values may be used by the analytics system to assess aspects of the patient's sleep. For example, time of onset of sleep may be measured by measuring electrical activity in the brain using one or more EEG sensors 418, as the electrical activity signals are different when a patient is awake and when the patient is asleep. Sleep efficiency may be measured using various mathematical formulae that include a ratio of total sleep time to time spent in bed along with many other factors affecting sleep as well as sleep discontinuity. For example, sleep efficiency %=(total sleep time/total time in bed)×100. Other sleep indices may be measured from the gathered data including but not limited to sleep latency, REM latency, wake after sleep onset, time and percentage in each sleep stage, etc. Breathing irregularities may be measured by using measurements from either chest palpitation sensor 416 or ECG sensor 406. Arousals may be measured using EEG 418, and cardiac rhythm abnormalities may be measured using ECG sensor 406. The accelerometer 412 and/or gyroscope 414 may measure body position as well as body movement of the patient during sleep. The oxygen saturation levels measured by using pulse oxygen sensor 410 integrated in the first wearable device 402 or oxygen sensor 420 integrated in the second wearable device 424 may also indicate breathing problems during sleep.

For example, normal blood oxygen level is generally 96-97%. Any dips in oxygen saturation measured by pulse oxygen sensor 410 or oxygen sensor 420 below this level may indicate breathing problems during the patient's sleep. As oxygen saturation in blood decreases, carbon dioxide levels in the blood increase, forcing the heart to pump more and more blood to oxygenate the blood. This activity also simultaneously causes the patient to breathe faster, which may be measured by microphone tracheal sensor or acoustic airway sensor 404, resulting in increased movement in the chest and abdomen and may be measured by chest palpitation sensors 416 and/or belt sensor 408. A continuous increase in the movement of the patient's chest and/or abdomen may result in the patient waking, also known as arousal, which may be detected by disruptive measurements taken by ECG sensor 406. This awakening along with the factors causing it may be recorded and quantified by the diagnostic components comprising data acquisition and analytics system to quantify the occurrence of such events during the sleep study, for example, number of awakenings related to dips in the oxygen saturation per hour, different stages of sleep, heart rate during different stages of sleep, respiratory disturbances during REM, NREM and/or MT events, and position of the patient and their correlation with different types of apnea such as obstructive apnea, central apnea, mixed apnea or hypopneas, etc.

In at least one embodiment, system 400 may include more than two wearable devices wherein each wearable device may include different or additional sensors than the ones described above. In at least one alternative embodiment, second wearable device 424 may be implemented as another type of device, for example, a wristband or a pendant.

Thus, FIG. 4 shows an exemplary embodiment of system 400 including, at least, first wearable device 402 having integrated therein a first set of sensors, second wearable device 424 comprising a second set of integrated sensors and memory HUB 422 that collects, stores, and or transmits data from any one or more of the sensors included in the first and second set of sensors.

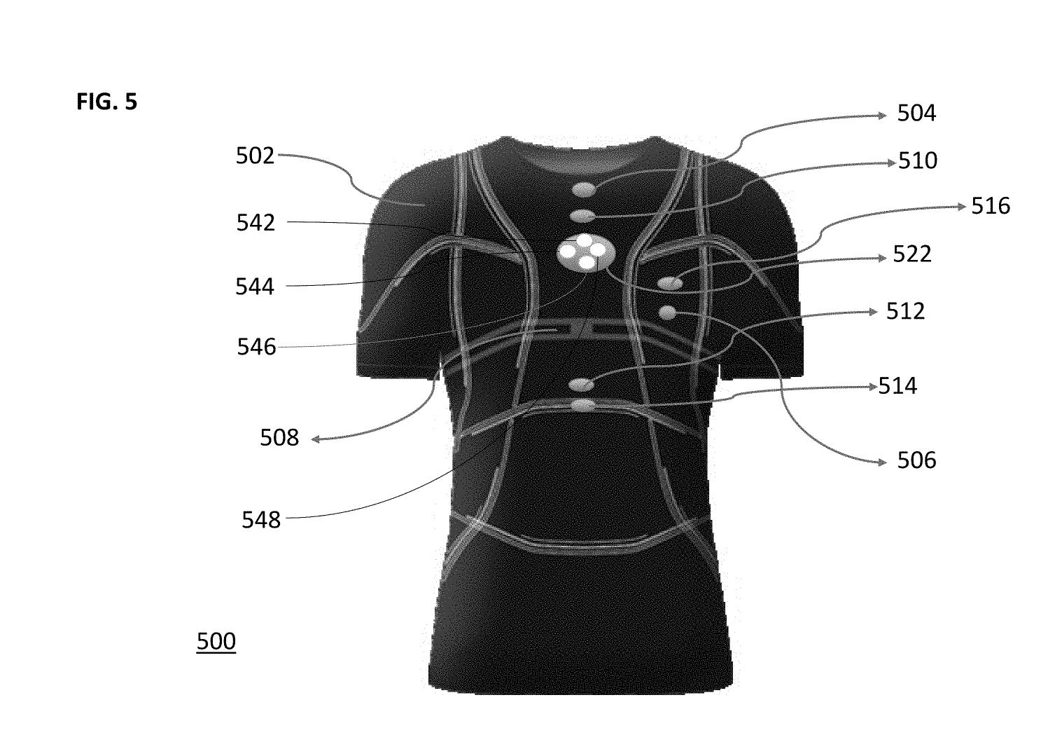

FIG. 5 shows an example first wearable device in which one or more embodiments of the system 300 or system 400 for conducting sleep study may be implemented, in accordance with various embodiments described herein. By way of example, but not limitation, configuration 500 includes first wearable device 502 that includes a first set of integrated sensors and memory HUB 522, also referred to as a HUB that receives and collects data from any one or more of the sensors included in the first and/or second set of sensors.

First wearable device 502 may be manufactured using a stretchable fabric for example, nylon, spandex, a combination of nylon and spandex, or Lycra. In an embodiment, the wearable device 502 may be a vest or a shirt made of stretchable fabric that may be pulled over reducing a need for a zipper or buttons.

The first set of sensors used to conduct the sleep study may be integrated into the first wearable device 502, in an unobtrusive manner, unlike wires of conventional sleep study machinery, which can be entangled with the sleep study patient or, at least, cause noticeable physical discomfort. In an embodiment, the first set of sensors can be integrated in the first wearable device by adhering the sensors to the material of the wearable device, for example, by using an adhesive, adhesive strips or using fabric fasteners such as Velcro or stitching or using sensors that are woven into the fabric of the wearable device. In an example embodiment, the wires to the sensor may be woven through the fabric. In another example embodiment, the fabric itself may have stretch sensors within the fabric that collect and transmit data.

The first set of integrated sensors as depicted may include any of a microphone tracheal sensor or acoustic airway sensor 504, electro cardiograph (ECG) sensor 506, belt sensor 508, as well as a pulse oxygen sensor 510, accelerometer 512, a gyroscope 514, and a chest palpitation sensor 516. These sensors are placed on the wearable device so as to enable sensing data from appropriate organs of a patient undergoing the study, for example, an ECG sensor 506 may be placed on the part of the first wearable device that is close to the patient's heart, a microphone tracheal sensor 504 may be placed close to the thoracic cavity or the trachea of the patient.

Microphone tracheal sensor or acoustic airway sensor 504 measures airflow by listening and recording airway sounds such as in the trachea (or windpipe) of the patient wearing, at least, first wearable device 502, including but not limited to snoring and apnea.

ECG sensor 506 records electrical activity of the heart over a period of time and is a non-gel contact sensor. Thus avoiding sticking sensors to a patient's body due to concerns of, e.g., skin allergies and discomfort associated with skin peeling. Examples of the recorded electrical activity include the subject's or patient's heart rate over a predetermined period of time or at predetermined intervals during the subject's or patient's sleep.

Belt sensor 508 detects, measures, and records chest expansion as the subject or patient wearing the wearable device sleeps. These belt sensors may be stretch sensors that have an electrical distortion sensor. The stretch sensors are displaced by expansion of the chest. As the belt sensor is pulled apart from its baseline resting position, it creates an electrical effect that is transmitted to the sensor hub. The signals may then be analyzed for extent of distortion and thereby the amount of chest expansion. These sensors record the amount of chest displacement in millimeters. This data may then be analyzed along with the data gathered by using pulse oximetry, and other vital information to determine the relationship between the person's breathing and their level of oxygenation.

Pulse oxygen sensor 510 monitors and records oxygen saturation of the patient's blood and changes in blood volume in the skin. This sensor monitors and records changes in blood oxygenation by analyzing the difference in transmission of two wavelengths of light that are sent through a diode light source in contact with the patient's skin. The difference in light transmission that is recorded may be calibrated and compared to normal standards resulting in a graphic or numerical value of blood oxygenation.

Accelerometer 512 and gyroscope 514 monitor and record sleeping position, changes in sleeping position, and stationary orientations of the patient while sleeping. An accelerometer is an electromechanical device that is used to measure acceleration and/or rotational movement. A gyroscope measures rotational movement as well. These mechanical forces are converted into electrical events that may be transmitted, recorded and analyzed by the software application. Positions described may include supine, left lateral, right lateral or prone.

Chest palpitation sensor 516 monitors and records the heart's electrical activity similar to ECG, by monitoring the heart's rhythm.

Memory HUB 522 may include a rechargeable power source 542, a removable storage device 544 for data collection, for example, a fob, a USB drive or a SD card, and a radio-frequency identification (RFID) node 546 to facilitate communication with any of the sensors integrated within first wearable device 502 and, optionally, second wearable device as depicted in FIG. 4. In at least one embodiment, memory HUB 522 may be adhered, stitched, or otherwise attached to the outer side of wearable device 502, may have a slim and non-intrusive form factor so as to minimize discomfort to the patient, and may include one or more sensors 548 that determine body position of the patient.

Body position sensor 548 determines body position of the patient using accelerometer 512. An accelerometer is an electromechanical device that is used to measure acceleration and/or rotational movement. These mechanical forces are then converted into electrical events that may be transmitted, recorded and analyzed by the software. Positions described may include supine, left lateral, right lateral or prone. RFID node 546 facilitates Bluetooth connectivity, or some other known RFID communication technology, with any of the sensors integrated within first wearable device 502 and optionally within the second wearable device 424 depicted in FIG.4. RFID technologies facilitating such communication may be implemented by, e.g., adding a RFID tag to any one or more sensors included in the first and/or second set of sensors for automatic identification and data capture working in concert with RFID node 546 included in memory HUB 522 or a smartphone.

In at least one other embodiment, memory HUB 522 may also include an interface for interacting with at least one mobile application that utilizes sleep analytics and trending features, for example, arousals due to breathing problems related to particular sleeping positions, arousals due to completion of sleep cycles, to provide a patient wearing any of the wearable devices described herein with information about his/her sleep patterns. The mobile application and/or its user interface may be hosted on a smartphone, tablet, PC, or other processor-driven computing device that is local to the patient. In an embodiment, the sensors may connect wirelessly or via Bluetooth with a smartphone application for data collection and transmission.

In at least one other embodiment, memory HUB 522 may itself host and/or operate a web based application to present the data collected by memory HUB 522 in a standardized format and to enable multi-day sleep studies for diagnosis using full trending and analytics. In an embodiment, the web application and/or its user interface may be hosted on a smartphone, tablet, PC, or other processor-driven computing device that may or may not be local to the patient.

In at least one other embodiment, memory HUB 522 may include a wireless communication node to upload the data collected by memory HUB 522 to a cloud database for live monitoring. The live monitoring may be achieved by communicatively coupling memory HUB 522 to at least one sensor of the first wearable device 502. In an embodiment, live monitoring may be implemented by communicatively coupling the cloud database to at least one sensor of the first wearable device 502 or to the memory HUB 522.

HUB 522 may include diagnostic components to record and quantify various aspects of a sleep study, e.g. different stages of sleep, heart rate during different stages of sleep, respiratory disturbances during REM, NREM and/or MT events, and position of the patient and their correlation with different types of apnea such as obstructive apnea, central apnea, mixed apnea or hypopneas. The diagnostic components may record and quantify data gathered by sensors including pulse oximetry, body position, chest movement, EEG and other sensors described above. Examples of the various aspects of comprehensive home-studied sleep including any of time of onset of sleep, sleep efficiency, sleep latency, REM latency, wake after sleep onset, time and percentage in each sleep stage, any breathing irregularities, for example, apneas which are defined as temporary cessation of breathing especially during sleep, arousals, cardiac rhythm abnormalities, body position, oxygen saturation and a combination thereof.

Once the data regarding these measurements over time during a sleep study is acquired, any diversions from the expected values may be used by the analytics system to provide diagnosis. For example, time of onset of sleep may be measured by measuring electrical activity in the heart using one or more ECG sensors 506, as the electrical activity signals are different when a patient is awake and when the patient is asleep. Sleep efficiency may be measured using various mathematical formulae that include a ratio of total sleep time to time spent in bed along with many other factors affecting sleep as well as sleep discontinuity. For example, sleep efficiency %=(total sleep time/total time in bed)×100. Other sleep indices may be measured from the gathered data including but not limited to sleep latency, REM latency, wake after sleep onset, time and percentage in each sleep stage, etc. Breathing irregularities may be measured by using measurements from either chest palpitation sensor 516 or ECG sensor 506. Arousals and cardiac rhythm abnormalities may be measured using ECG sensor 506. The accelerometer 512 and/or gyroscope 514 may determine body position as well as measure body movement of the patient during sleep. The oxygen saturation levels measured by using pulse oxygen sensor 510 integrated in the first wearable device 502 may also indicate breathing problems during sleep.

For example, normal blood oxygen level is generally 96-97%. Any dips in oxygen saturation detected and/or measured by pulse oxygen sensor 510 below this level may indicate breathing problems during the patient's sleep. As oxygen saturation in blood decreases, carbon dioxide levels in the blood increase, forcing the patient's heart to pump increasing amounts of blood to oxygenate the blood. This activity also simultaneously causes the patient to breathe faster and increased airflow which may be measured by microphone tracheal sensor or acoustic airway sensor 504, resulting in increased movement in the chest and abdomen and may be measured by chest palpitation sensors 516 and/or belt sensor 508. The continuous increase in the movement of chest and abdomen may result in awakening the patient also known as arousal which may be measured by ECG sensor 506. This awakening along with the factors causing it may be recorded and quantified by the diagnostic components comprising data acquisition and analytics system to quantify the occurrence of such events during the sleep study, for example, number of awakenings related to dips in the oxygen saturation per hour, different stages of sleep, heart rate during different stages of sleep, respiratory disturbances during REM, NREM and/or MT events, and position of the patient and their correlation with different types of apnea such as obstructive apnea, central apnea, mixed apnea or hypopneas, etc.

Thus, FIG. 5 shows an exemplary embodiment 500 of wearable device 502, having at least one set of integrated sensors including any of microphone tracheal sensor or acoustic airway sensor 504, electro cardiograph (ECG) sensor 506, belt sensor 508, pulse oxygen sensor 510, accelerometer 512, gyroscope 514, chest palpitation sensor 516 and a combination thereof; and a memory HUB 522 that collects data from any one or more sensors included in the at least one set of integrated sensors. The set of sensors monitors sleep cycles of a patient, records data and transmits the recorded data to memory HUB 522.

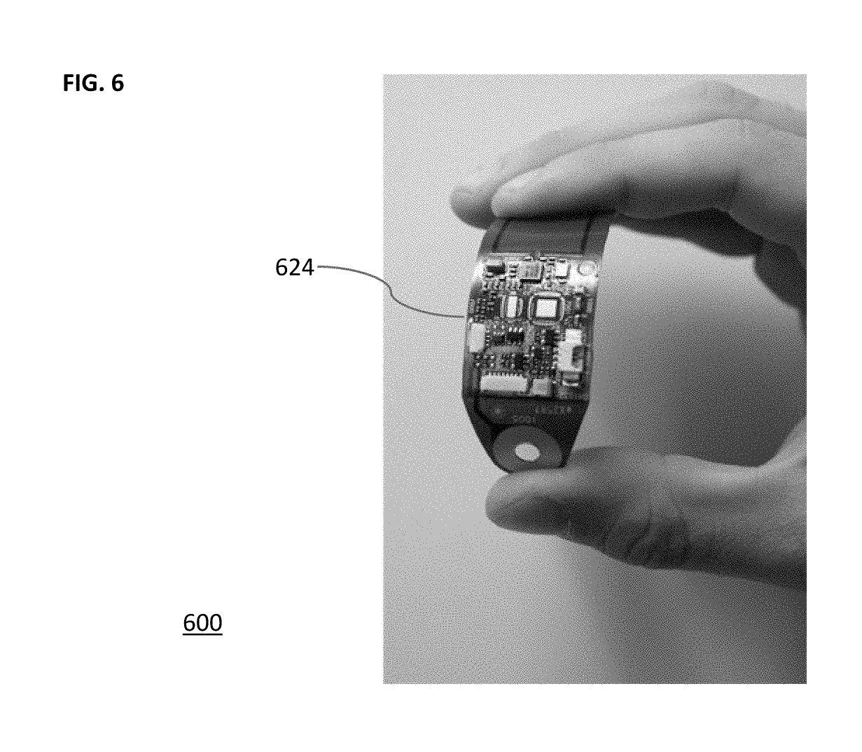

FIG. 6 shows an example second wearable device in which one or more embodiments of the system 400 for conducting sleep study may be implemented, in accordance with various embodiments described herein. The second wearable device 624 may be implemented as, for example, a headband or a cap, comprising a second set of integrated sensors. In an embodiment, the second set of sensors may be integrated in the second wearable device by adhering the sensors to the material of the wearable device, for example, by using an adhesive, adhesive strips or using fabric fasteners such as Velcro or stitching or using sensors that are woven into the fabric of the wearable device. In an example embodiment, the wires to the sensor may be woven through the fabric. In another example embodiment, the fabric itself may have stretch sensors within the fabric that collect and transmit data.

The second set of integrated sensors as depicted may include any of one or more electroencephalogram (EEG) sensors 618 and an oxygen sensor 620. The one or more EEG sensors 618 monitor and record electrical activity in the brain and the oxygen sensor 620 monitors and records oxygen saturation in human blood non-invasively through skin. The one or more EEG sensors measure voltage fluctuations resulting from ionic current within the neurons of the brain. These voltage fluctuations may be amplified, recorded and analyzed by the software application. To record the patient's brain activity during the sleep study, the one or more EEG sensors 618 may be placed on a headband or a cap that may fit closer to the patient's scalp. Similarly, to monitor and record oxygen saturation in human blood non-invasively through skin, the oxygen sensor 620 may be placed close to the skin on the patient's forehead. Implementation of the system may avoid attaching any devices or sensors to any part of the mouth or nose, which could impede or otherwise disrupt the patient's sleep. In an embodiment, one or more sensors included in the second set of sensors may communicate either with memory HUB 522 attached to first wearable device, for example, the device 502 illustrated in FIG. 5 or to a smartphone application directly.

Thus FIG. 6 shows an exemplary embodiment 600 of the second wearable device 624 comprising a second set of integrated sensors.

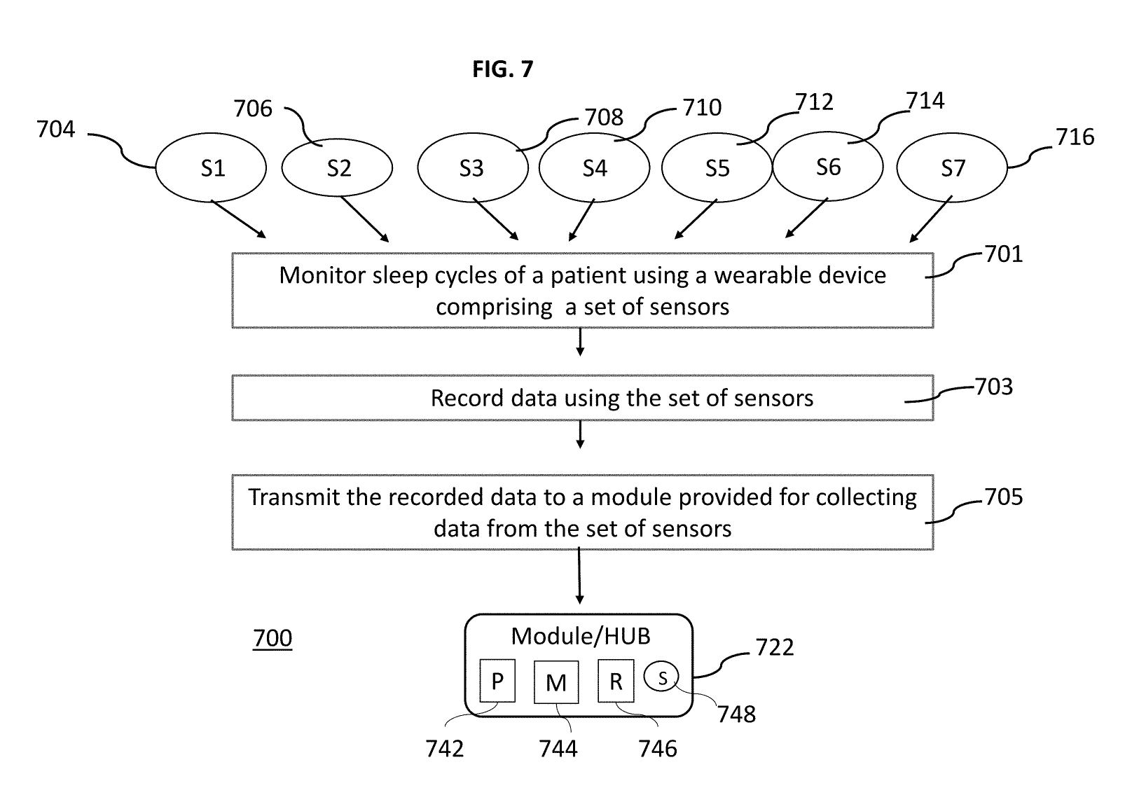

FIG. 7 shows an example processing flow of operations for implementing at least portions of a system for conducting sleep study using at least one wearable processor-enabled device, in accordance with various embodiments described herein. In at least one embodiment, the at least one wearable processor-enabled device includes at least one set of sensors and a memory HUB as described in detail in the description accompanying FIG. 5.

By way of example, but not limitation, the process for conducting sleep study using one or more wearable processor-enabled devices includes monitoring sleep cycles of at least one patient wearing the processor-enabled device via step 701, and by recording data via step 703 over a period of time and/or at predetermined time intervals using at least one set of sensors, for example, any of S1 704, S2 706, S3 708, S4 710, S5 712, S6 714 and S7 716, or a combination of any of these sensors. The recorded data is then transmitted via step 705 to a memory HUB 722 that collects data from one or more sensors from the at least one set of sensors. One or more sensors included in the at least one set of sensors communicates with the memory HUB 722 for collecting data by Bluetooth, or some other known RFID transmission. The possible RFID technologies may include adding a RFID tag to any one or more sensors included in the at least one set of sensors for automatic identification and data capture working in concert with the RFID node 746 included in the memory HUB 722 or a smartphone.

In an example embodiment, the process of monitoring sleep cycle of the patient is executed by a first set of sensors, for example, any of S1 704, S2 706, S3 708, S4 710, S5 712, S6 714 and S7 716, or a combination of any of these sensors, integrated within first wearable processor-enabled device, for example, device 502 shown in FIG. 5. In an embodiment, the first set of sensors may include any of microphone tracheal sensor or acoustic airway sensor, electro cardiograph (ECG) sensor, belt sensor, accelerometer, gyroscope, chest palpitation sensor and a combination thereof. The ECG sensor records electrical activity of the heart over a period of time and is a non-gel contact sensor. Thus avoiding sticking sensors to a patient's body due to concerns around skin allergies and discomfort associated with skin peeling. The belt sensor records chest expansion. The pulse oxygen sensor monitors and records oxygen saturation of the patient's blood and changes in blood volume in the skin. The accelerometer and gyroscope monitor and record position and orientation of the patient, and the chest palpitation sensor monitors and records the heart's electrical activity similar to ECG. Working and functions of various sensors enumerated here are described in detail in the descriptions accompanying FIG. 5.

In an exemplary embodiment, memory HUB or HUB 722 that collects data may be removably attached to the first wearable processor-enabled device and comprises a rechargeable power source 742, a removable storage device 744, for example, a fob, a USB drive or a SD card to collect data from sensors integrated within the wearable processor-enabled device, and an RFID node 746 to facilitate communication with the at least one set of sensors. In an embodiment the memory HUB or HUB 722 may be situated on the outside of the wearable device, may be slim and non-intrusive and may include one or more sensors 748 that determine body position of the patient. The body position sensor 748 may detect movement and/or determine the patient's current body position using an accelerometer. An accelerometer is an electromechanical device that is used to measure acceleration and/or rotational movement. These mechanical forces are then converted into electrical events that may be transmitted, recorded and analyzed by the software application. Positions described may include supine, left lateral, right lateral or prone.

In an embodiment, the process may further implement instructions for memory HUB 722 to interact with at least one mobile application via a mobile application interface. The mobile application may utilize sleep analytics and trending features, for example, arousals due to breathing problems related to particular sleeping positions, arousals due to completion of sleep cycles, to provide a patient wearing the wearable device with information about their sleep patterns. In an embodiment, the sensors may connect wirelessly or via Bluetooth with a smartphone application for data collection and transmission. The smartphone application and/or its user interface may be hosted on a smartphone, tablet, PC, or other processor-driven computing device that is local to the patient.

In another embodiment, the process may further include instructions for the memory HUB 722 to interact with a web based application via wireless communication node to present the data collected by the memory HUB 722 in a standardized format and to enable multi-day sleep studies for diagnosis using full trending and analytics. In an embodiment, the web based application and/or its user interface may be hosted on a smartphone, tablet, PC, or other processor-driven computing device that may or may not be local to the patient. In another embodiment, the web based application may be hosted on the memory HUB 722 itself.

In yet another embodiment, the process may further include instructions for live monitoring of a patient's sleep cycles by uploading the data collected by the memory HUB 722 to a cloud database via wireless communication. The live monitoring may be implemented by communicatively coupling one or more sensors of at least one of the one or more wearable processor-enabled device to the memory HUB 722. In an embodiment, the live monitoring may be achieved by communicatively coupling the cloud database to one or more sensors of the wearable processor-enabled devices or to memory HUB 722.

The executable instructions for monitoring sleep cycles of the patient may further instruct the sensors and/or memory HUB to record and quantify standardized aspects of home-studied sleep using one or more diagnostic metrics, e.g. different stages of sleep, heart rate during different stages of sleep, respiratory disturbances during REM, NREM and/or MT events, and position of the patient and their correlation with different types of apnea such as obstructive apnea, central apnea, mixed apnea or hypopneas. The diagnostic metrics may be determined by recording and quantifying standardized aspects of comprehensive home-studied sleep including any of time of onset of sleep, sleep efficiency, sleep latency, REM latency, wake after sleep onset, time and percentage in each sleep stage, any breathing irregularities, for example, apneas which are defined as temporary cessation of breathing especially during sleep, arousals, cardiac rhythm abnormalities, body position, oxygen saturation and a combination thereof.

Once the data regarding these measurements over time during a sleep study is acquired, any diversions from the expected values may be used by the analytics system to provide diagnosis. For example, time of onset of sleep may be measured by measuring electrical activity in the heart using one or more ECG sensors 706, as the electrical activity signals are different when a patient is awake and when the patient is asleep. Sleep efficiency may be measured using various mathematical formulae that include a ratio of total sleep time to time spent in bed along with many other factors affecting sleep as well as sleep discontinuity. For example, sleep efficiency %=(total sleep time/total time in bed)×100. Other sleep indices may be measured from the gathered data including but not limited to sleep latency, REM latency, wake after sleep onset, time and percentage in each sleep stage etc. Breathing irregularities may be measured by using measurements from either chest palpitation sensor 716 or ECG sensor 706. Arousals and cardiac rhythm abnormalities may be measured using ECG sensor 706. The accelerometer 712 and/or gyroscope 714 may determine body position as well as measure body movement of the patient during sleep. The oxygen saturation levels measured by using pulse oxygen sensor 710 integrated in the first wearable device may also indicate breathing problems during sleep.

As set forth below, dips in oxygen saturation below 96 or 97% detected and/or measured by pulse oxygen sensor 710 may indicate breathing problems during the patient's sleep. As oxygen saturation in blood decreases, carbon dioxide levels in the blood increase, forcing the heart to pump more and more blood to oxygenate the blood. This activity also simultaneously causes the patient to breathe faster resulting in increased airflow which may be measured by microphone tracheal sensor or acoustic airway sensor 704, resulting in increased movement in the chest and abdomen and may be measured by chest palpitation sensors 716 and/or belt sensor 708. The continuous increase in the movement of chest and abdomen may result in awakening the patient also known as arousal which may be measured by ECG sensor 706. This awakening along with the factors causing it may be recorded and quantified by the diagnostic components comprising data acquisition and analytics system to quantify the occurrence of such events during the sleep study, for example, number of awakenings related to dips in the oxygen saturation per hour, different stages of sleep, heart rate during different stages of sleep, respiratory disturbances during REM, NREM and/or MT events, and position of the patient and their correlation with different types of apnea such as obstructive apnea, central apnea, mixed apnea or hypopneas, etc.

Thus, the method for performing a comprehensive sleep study using at least one processor-enabled wearable devices includes monitoring sleep cycles of a patient wearing the at least one processor-enabled devices having at least one set of sensors, by recording data using the at least one set of sensors including, at least: airway sounds, including snoring and apnea, and electrical activity of the patient's heart over a period of time; and receiving the recorded data by a memory HUB by Bluetooth transmission; wherein any one or more of the sensors included in the at least one set of sensors communicates with the memory HUB that collects data from the at least one set of sensors.

Thus, FIG. 7 shows an example of the process for conducting sleep study using at least one wearable processor-enabled device including monitoring sleep cycles of at least one patient wearing the processor-enabled device.

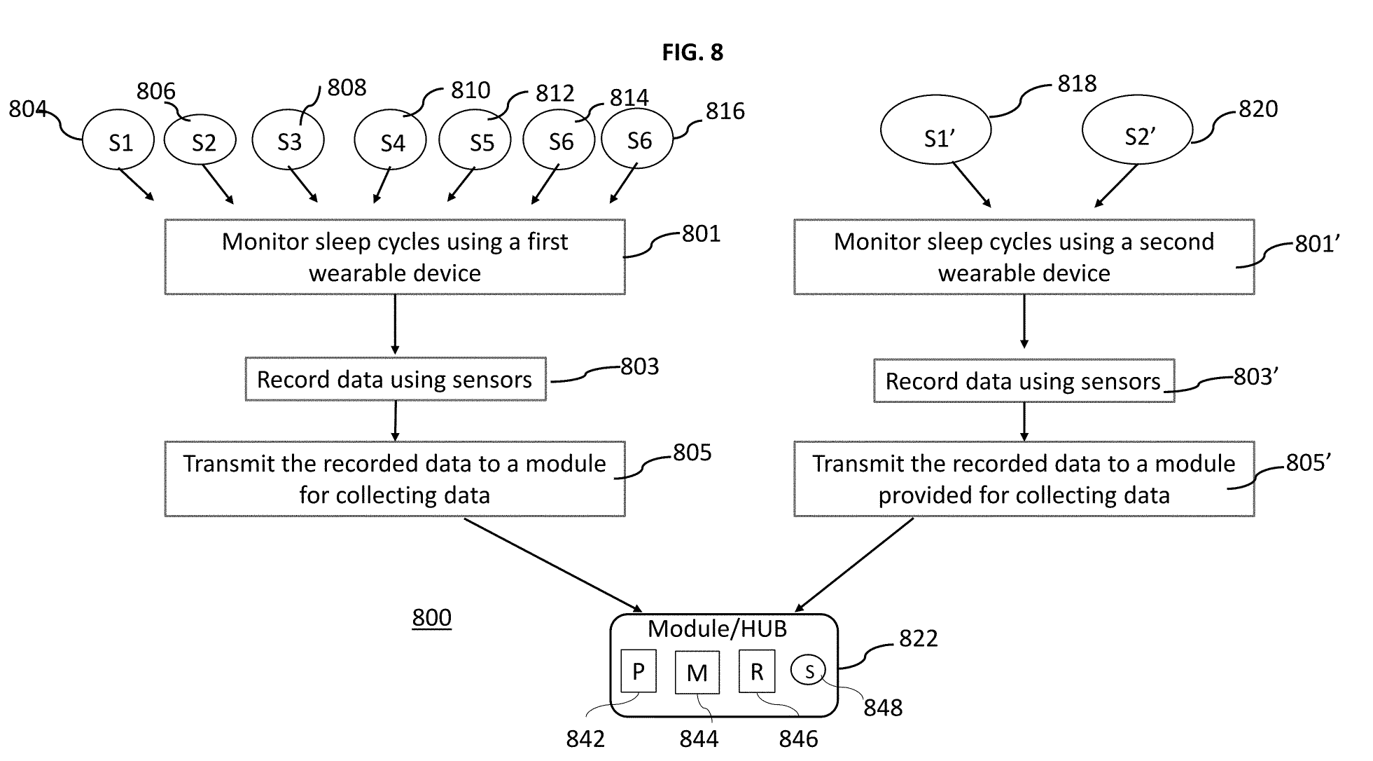

FIG. 8 shows an example processing flow of operations for implementing at least portions of a system for conducting sleep study using two wearable processor-enabled devices, in accordance with various embodiments described herein. The process for conducting sleep study using two wearable processor-enabled devices includes monitoring sleep cycles of at least one patient wearing a first processor-enabled device via step 801 by recording data via step 803 over a period of time by using a first set of sensors, for example, any of S1 804, S2 806, S3 808, S4 810, S5 812, S6 814 and S7 816, or a combination of any of these sensors; and a second processor-enabled device via step 801′ by recording data via step 803′ over a period of time by using a second set of sensors, for example, any of S1′ 818, S2′ 820, or a combination of these sensors. The data recorded by the first set of sensors is then transmitted via step 805 to a memory HUB 822 that collects data from one or more sensors from the first set of sensors and the second set of sensors. Similarly, the data recorded by the second set of sensors is transmitted via step 805′ to a memory HUB 822 that collects data from one or more sensors from the first set of sensors and the second set of sensors. At least one sensor from the first set of sensors and the second set of sensors communicates with the memory HUB 822 for collecting data by Bluetooth or some other known RFID transmission. The possible RFID technologies may be implemented by attaching or otherwise associating an RFID tag to or with any sensor of the first set of sensors and the second set of sensors for automatic identification and data capture working in concert with RFID node 846 included in memory HUB 822 or a smartphone.

In at least one embodiment, the first wearable processor-enabled device includes a first set of sensors and the second wearable processor-enabled device includes a second set of sensors as described in detail in the descriptions accompanying FIG. 5 and FIG. 6.

In an example embodiment, the first set of sensors may include any of microphone tracheal sensor or acoustic airway sensor, electro cardiograph (ECG) sensor, belt sensor, accelerometer, gyroscope, chest palpitation sensor and a combination thereof. The ECG sensor records electrical activity of the heart over a period of time and is a non-gel contact sensor. Thus avoiding sticking sensors to a patient's body due to concerns around skin allergies and discomfort associated with skin peeling. The belt sensor records chest expansion. The pulse oxygen sensor monitors and records saturation of the patient's blood and changes in blood volume in the skin. The accelerometer and gyroscope monitor and record position and orientation of the patient, and the chest palpitation sensor monitors and records the heart's electrical activity similar to ECG. Working and functions of various sensors included in the first set of sensors enumerated here are described in detail in the descriptions accompanying FIG. 5.

In an embodiment, the executable instructions for monitoring sleep cycles of the at least one patient are executed, additionally or alternatively, by a second set of sensors integrated within the second wearable device of the one or more processor-enabled devices, the second set of sensors including any of one or more electroencephalogram (EEG) sensors 818, an oxygen sensor 820, or a combination thereof, along with the first set of sensors as described above and shown in FIG. 8. Working and functions of various sensors included in the second set of sensors enumerated here are described in detail in the descriptions accompanying FIG. 6.

In another embodiment, the process of monitoring sleep cycles may be executed by more than two wearable processor enabled devices, wherein each wearable device may include different or additional sensors than the ones described above. In an embodiment the second wearable may be another type of device, for example, a wristband or a pendant.

In an exemplary embodiment, memory HUB 822 that collects data is removably attached to the first wearable processor-enabled device and includes rechargeable power source 842, removable storage device 844, for example, a fob, a USB drive or a SD card to collect data from sensors integrated within the wearable processor-enabled device, and RFID node 846 to facilitate communication with the at least one set of sensors. In an embodiment memory HUB 822 may be situated on the outside of the wearable device, may be slim and non-intrusive and may include one or more sensors 848 that determine body position of the patient. Body position sensor 848 may detect movement of the patient while sleeping and determine a current body position of the patient at any given time using an accelerometer. An accelerometer is an electromechanical device that is used to measure acceleration and/or rotational movement. These mechanical forces are then converted into electrical events that may be transmitted, recorded and analyzed by the software. Positions described may include supine, left lateral, right lateral or prone.

In an embodiment, the process may further comprise instructions for the memory HUB 822 to interact with at least one mobile application via mobile application interface. The mobile application may utilize sleep analytics and trending features, for example, arousals due to breathing problems related to particular sleeping positions, arousals due to completion of sleep cycles, to provide a patient wearing the wearable device with information about their sleep patterns. In an embodiment, the sensors may connect wirelessly or via Bluetooth with a smartphone application for data collection and transmission. The smartphone application and/or its user interface may be hosted on a smartphone, tablet, PC, or other processor-driven computing device that is local to the patient.

In at least one other embodiment, the process may further include instructions for the memory HUB 822 to interact with a web based application via wireless communication node to present the data collected by the memory HUB 822 in a standardized format and to enable multi-day sleep studies for diagnosis using full trending and analytics. In an embodiment, the web based application and/or its user interface may be hosted on a smartphone, tablet, PC, or other processor-driven computing device that may or may not be local to the patient. In another embodiment, the web based application may be hosted on the memory HUB 822 itself.

In an embodiment, the process may further include instructions for live monitoring of a patient's sleep cycles by uploading the data collected by the memory HUB 822 to a cloud database via wireless communication. The live monitoring may be achieved by communicatively coupling the memory HUB 822 to at least one sensor of the first wearable device. In an embodiment, the live monitoring may be achieved by communicatively coupling the cloud database to one or more sensors of at least one of the first wearable device and the second wearable device, or to the memory HUB 822.

The executable instructions for monitoring sleep cycles of the patient may further include instructions for recording and quantifying of standardized aspects of home-studied sleep using one or more diagnostic metrics, e.g. different stages of sleep, heart rate during different stages of sleep, respiratory disturbances during REM, NREM and/or MT events, and position of the patient and their correlation with different types of apnea such as obstructive apnea, central apnea, mixed apnea or hypopneas. The diagnostic metrics may be determined by recording and quantifying standardized aspects of comprehensive home-studied sleep including any of time of onset of sleep, sleep efficiency, sleep latency, REM latency, wake after sleep onset, time and percentage in each sleep stage, any breathing irregularities, for example, apneas which are defined as temporary cessation of breathing especially during sleep, arousals, cardiac rhythm abnormalities, body position, oxygen saturation and a combination thereof.

Once the data regarding these measurements over time during a sleep study is acquired, any diversions from the expected values may be used by the analytic system to provide diagnosis. For example, time of onset of sleep may be measured by measuring electrical activity in the brain using one or more EEG sensors 818, as the electrical activity signals are different when a patient is awake and when the patient is asleep. Sleep efficiency may be measured using various mathematical formulae that include a ratio of total sleep time to time spent in bed along with many other factors affecting sleep as well as sleep discontinuity. For example, sleep efficiency %=(total sleep time/total time in bed)×100. Other sleep indices may be measured from the gathered data including but not limited to sleep latency, REM latency, wake after sleep onset, time and percentage in each sleep stage etc. Breathing irregularities may be measured by using measurements from either chest palpitation sensor 816 or ECG sensor 806. Arousals may be measured using EEG sensor 818, and cardiac rhythm abnormalities may be measured using ECG sensor 806. Accelerometer 812 and/or gyroscope 814 may measure body position as well as body movement of the patient during sleep. The oxygen saturation levels measured by using pulse oxygen sensor 810 integrated in the first wearable device or oxygen sensor 820 integrated in the second wearable device may also indicate breathing problems during sleep.

For example, noticeable drops in oxygen saturation measured by pulse oxygen sensor 810 or oxygen sensor 820 may indicate breathing problems during the patient's sleep. As oxygen saturation in blood decreases, carbon dioxide levels in the blood increase, forcing the heart to pump more and more blood to oxygenate the blood. This activity also simultaneously causes the patient to breathe faster resulting in increased airflow which may be measured by microphone tracheal sensor or acoustic airway sensor 804, resulting in increased movement in the chest and abdomen and may be measured by chest palpitation sensors 816 and/or belt sensor 808. The continuous increase in the movement of chest and abdomen may result in awakening the patient also known as arousal which may be measured by ECG sensor 806. This awakening along with the factors causing it may be recorded and quantified by the diagnostic components comprising data acquisition and analytics system to quantify the occurrence of such events during the sleep study, for example, number of awakenings related to dips in the oxygen saturation per hour, different stages of sleep, heart rate during different stages of sleep, respiratory disturbances during REM, NREM and/or MT events, and position of the patient and their correlation with different types of apnea such as obstructive apnea, central apnea, mixed apnea or hypopneas, etc.

Thus, the method for performing a comprehensive sleep study using two processor-enabled wearable devices includes monitoring sleep cycles of a patient wearing the two processor-enabled devices having at least one set of sensors, by recording data using the at least one set of sensors including: airway sounds, including snoring and apnea, and electrical activity of the patient's heart over a period of time; and receiving the recorded data by a memory HUB by Bluetooth transmission; wherein any one or more of the sensors included in the at least one set of sensors communicates with the memory HUB that collects data from the at least one set of sensors.

Thus, FIG. 8 shows an example processing flow of operations for implementing at least portions of a system for conducting sleep study using two wearable processor-enabled devices, in accordance with various embodiments described herein.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In an illustrative embodiment, any of the operations, processes, etc. described herein can be implemented as computer-readable instructions stored on a computer-readable medium. The computer-readable instructions can be executed by a processor of a mobile unit, a network element, and/or any other computing device.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

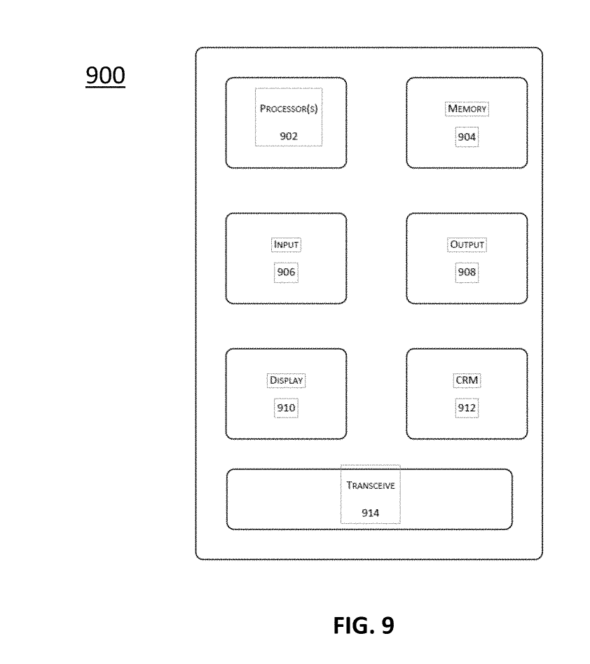

FIG. 9 shows a block diagram illustrating an example computing device by which various embodiments of the process of conducting sleep study described herein may be implemented.

More particularly, FIG. 9 shows an illustrative computing embodiment, in which any of the processes and sub-processes for conducting a sleep study may be implemented as computer-readable instructions stored on a computer-readable medium. The computer-readable instructions may, for example, be executed by a processor of a device, as referenced herein, having a network element and/or any other device corresponding thereto, particularly as applicable to the applications and/or programs described above corresponding to the configuration 300 and 400 for transactional processes

In a very basic configuration, a computing device 900 may typically include, at least, one or more processors 902, a system memory 906, one or more input components 906, one or more output components 908, a display component 910, a computer-readable medium 912, and a transceiver 914.

Processor 902 may refer to, e.g., a microprocessor, a microcontroller, a digital signal processor, or any combination thereof.

Memory 904 may refer to, e.g., a volatile memory, non-volatile memory, or any combination thereof. Memory 904 may store, therein, an operating system, an application, and/or program data. That is, memory 904 may store executable instructions to implement any of the functions or operations described above and, therefore, memory 904 may be regarded as a computer-readable medium.

Input component 906 may refer to a built-in or communicatively coupled keyboard, touch screen, or telecommunication device. Alternatively, input component 906 may include a microphone that is configured, in cooperation with a voice-recognition program that may be stored in memory 904, to receive voice commands from a user of computing device 900. Further, input component 906, if not built-in to computing device 900, may be communicatively coupled thereto via short-range communication protocols including, but not limited to, radio frequency or Bluetooth.

Output component 908 may refer to a component or memory HUB, built-in or removable from computing device 900, that is configured to output commands and data to an external device.

Display component 910 may refer to, e.g., a solid state display that may have touch input capabilities. That is, display component 910 may include capabilities that may be shared with or replace those of input component 906.

Computer-readable medium 912 may refer to a separable machine readable medium that is configured to store one or more programs that embody any of the functions or operations described above. That is, computer-readable medium 912, which may be received into or otherwise connected to a drive component of computing device 900, may store executable instructions to implement any of the functions or operations described above. These instructions may be complimentary or otherwise independent of those stored by memory 904.

Transceiver 914 may refer to a network communication link for computing device 900, configured as a wired network or direct-wired connection. Alternatively, transceiver 914 may be configured as a wireless connection, e.g., radio frequency (RF), infrared, Bluetooth, and other wireless protocols.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

We claim:

1. A system for performing a comprehensive sleep study, comprising:
at least one wearable device comprising at least one set of integrated sensors; and
a memory HUB for receiving and storing data from any one or more of the sensors included in the at least one set of sensors;
wherein the any one or more of the sensors included in the at least one set of sensors monitor sleep cycles of a patient wearing at least one wearable device, record data, and transmit the recorded data to the memory HUB.

2. The system of claim 1, wherein the at least one wearable device comprises a first wearable device comprising a first set of integrated sensors including any of microphone tracheal sensor, electro cardiograph (ECG) sensor, belt sensor, pulse oxygen sensor, accelerometer, gyroscope, chest palpitation sensor and a combination thereof.

3. The system of claim 2, wherein the at least one wearable device further comprises a second wearable device comprising a second set of integrated sensors including any of one or more electroencephalogram (EEG) sensors, an oxygen sensor and a combination thereof.

4. The system of claim 2, wherein the microphone tracheal sensor measures airflow by recording airway sounds.

5. The system of claim 2, wherein the ECG sensor records electrical activity of a heart over a period of time and is a non-gel contact sensor.

6. The system of claim 2, wherein the belt sensor records chest expansion of the patient wearing at least the first wearable device.

7. The system of claim 2, wherein the first wearable device comprises a stretchable fabric and the one or more integrated sensors are woven in the stretchable fabric of the first wearable device.

8. The system of claim 1, wherein the memory HUB comprises a rechargeable power source, a removable device for data collection and storage, and a radio-frequency identification (RFID) node to facilitate communication with any one or more of the sensors included in the at least one set of sensors.

9. The system of claim 8, wherein the RFID node facilitates Bluetooth connectivity.

10. The system of claim 1, further comprising one or more diagnostic components including a data acquisition and data analytics system to record and quantify standardized aspects of home-studied sleep.

11. The system of claim 10, wherein the standardized aspects of home-studied sleep comprise any of time of onset of sleep, sleep efficiency, sleep latency, REM latency, wake after sleep onset, time and percentage in each sleep stage, breathing irregularities, apnea, arousals, abnormalities in cardiac rhythm, body position, oxygen saturation and a combination thereof.

12. The system of claim 8, wherein the memory HUB further comprises an interface for interacting with at least one mobile application that utilizes sleep analytics and trending features to provide a patient wearing the at least one wearable device with information about their sleep patterns.

13. The system of claim 1, further comprising a wireless communication node to upload the data collected by the memory HUB to a cloud database for live monitoring, wherein the cloud database is communicatively coupled to the at least one wearable device or to the memory HUB.

14. The system of claim 13, further comprising a web based application to present the data collected by the memory HUB in a standardized format and to enable multi-day sleep studies for diagnosis using full trending and analytics.

15. A non-transitory computer-readable medium having executable instructions stored therein that, when executed, cause one or more wearable processor-enabled devices performing a comprehensive sleep study and comprising at least one set of sensors to perform operations comprising:
monitoring sleep cycles of a patient wearing the one or more processor-enabled devices, by recording data including
airway sounds, including snoring and apnea, and
electrical activity of a user's heart over a period of time; and
transmitting the recorded data to a memory HUB that collects data from the at least one set of sensors;
wherein the at least one set of sensors communicates with the memory HUB by Bluetooth transmission.

16. The computer-readable medium of claim 15, wherein the executable instructions are executed, at least in part, by a first set of sensors integrated within a first wearable device of the one or more processor-enabled devices, the first set of sensors comprising any of microphone tracheal sensor, electro cardiograph (ECG) sensor, belt sensor, pulse oxygen sensor, accelerometer, gyroscope, chest palpitation sensor and a combination thereof.

17. The computer-readable medium of claim 15 wherein the ECG sensor is a non-gel contact sensor.

18. The computer-readable medium of claim 15, wherein the belt sensor records chest expansion of the patient.

19. The computer-readable medium of claim 15, wherein the first wearable processor-enabled device comprises a stretchable fabric and the integrated first set of sensors are woven in the stretchable fabric.

20. The computer-readable medium of claim 15, wherein the executable instructions are executed, at least in part, by a second set of sensors integrated within a second wearable device of the one or more processor-enabled devices, the second set of sensors comprising any of one or more electroencephalogram (EEG) sensors, an oxygen sensor, or a combination thereof.

21. The computer-readable medium of claim 15, wherein the memory HUB is removably attached to the first wearable processor-enabled device and comprises a rechargeable power source, a removable node to collect data from sensors integrated within the wearable processor-enabled device, and an RFID node to facilitate communication with any one or more of the sensors included in the at least one set of sensors.

22. The computer-readable medium of claim 15, wherein the operations further comprise recording and quantifying standardized aspects of home-studied sleep using one or more diagnostic metrics.

23. A wearable device for performing a comprehensive sleep study, comprising:
at least one set of integrated sensors including any of microphone tracheal sensor, electro cardiograph (ECG) sensor, belt sensor, pulse oxygen sensor, accelerometer, gyroscope, chest palpitation sensor and a combination thereof; and
a memory HUB that receives and stores data from any one or more of the sensors included in the at least one set of integrated sensors;
wherein the at least one set of sensors monitors sleep cycles of a patient wearing the wearable device, records data and transmits the recorded data to the memory HUB.

24. A method for performing a comprehensive sleep study using one or more processor-enabled wearable devices, comprising:
monitoring sleep cycles of a patient wearing the one or more processor-enabled devices comprising at least one set of sensors, by recording data using the at least one set of sensors including
airway sounds, including snoring and apnea, and
electrical activity of the patient's heart over a period of time; and
receiving the recorded data by a memory HUB by Bluetooth transmission;
wherein any one or more of the sensors included in the at least one set of sensors communicates with the memory HUB that collects data from the at least one set of sensors.
